(19) United States
(12) Patent Application Publication
(10) Pub. No.: US 2009/0171406 A1
(43) Pub. Date: Jul. 2, 2009

(54) ELECTRICALLY CONDUCTING SCAFFOLDS FOR CELL-BASED PACING

(75) Inventors: John Foley, Lino Lakes, MN (US); Julio C. Spinelli, Buenos Aires (AR)

Correspondence Address: SCHWEGMAN, LUNDBERG & WOESSNER/BSC-CRM, PO BOX 2938, MINNEAPOLIS, MN, 55402, US

(73) Assignee: Cardiac Pacemakers, Inc., st. paul, MN (US)

(21) Appl. No.: 12/328,591

(22) Filed: Dec. 4, 2008

Related U.S. Application Data
(60) Provisional application No. 61/013,208, filed on Dec. 12, 2007.

(51) Int. Cl. A61K 9/10; A61K 35/12; A61N 1/00

(52) U.S. Cl. 607/3; 424/93.7; 424/484; 424/93.21; 607/116

(57) ABSTRACT

The invention provides a biological electrode including a first biocompatible polymer, an electrically conducting biocompatible polymer and mammalian donor cells and an electrical stimulation system for delivering electrical stimulation into target tissue including target cells. The system includes a biological electrode including an electrically conductive polymeric matrix and mammalian donor cells embedded in the electrically conductive polymeric matrix.

### CROSS-REFERENCE TO RELATED APPLICATION

This application claims the benefit of U.S. Provisional Application No. 61/013,208, filed on Dec. 12, 2007, under 35 U.S.C. § 119(e), which is hereby incorporated by reference.

### BACKGROUND

Cardiovascular diseases are responsible for a preponderance of health problems in the majority of the developed countries as well as in many developing countries. Heart disease and stroke, the principal components of cardiovascular disease, are the first and third leading cause of mortality in the U.S., accounting for nearly 40% of all deaths (Heart and Stroke Statistical Update, American Heart Association 2002). Cardiovascular diseases also include congenital heart defects, which occur in about 1% of live births (Gillum, Am. Heart J., 127:919 (1994)) and are the main cause of mortality in the first year of life (Hoffman, Pediatr. Cardiol., 16:103 (1995)). When they do not lead to death, cardiovascular diseases may alternatively result in substantial disability and loss of productivity. About 61 million Americans (almost one-fourth of the population) live with cardiovascular disorders, such as coronary heart disease, congenital cardiovascular defects, and congestive heart failure. In 2001, 298.2 billion dollars were spent in the treatment of these clinical conditions, and the economic impact of cardiovascular disease on the U.S. health care system is expected to grow as the population ages.

Over the past 30 years, advances in the treatment and prevention of cardiac diseases have led to constantly declining morbidity and mortality rates. Treatments for both congenital heart defects and cardiomyopathies have become more sophisticated. However, when these treatments fail, organ or tissue replacement remains the only other possible option. Different surgical procedures may be performed to treat heart failure and cardiac deficiency. These procedures include transplantation of organs from one individual to another, reconstructive surgery, and implantation of mechanical devices such as mechanical heart valves.

Cardiac transplantation is so common that the primary limitation on patient outcome is not the surgical technique, but the declining availability of donor organs. In 2000, 2,500 heart transplants were performed in the U.S. while it was estimated that between 20,000 and 40,000 patients could have benefited from such a medical operation. To circumvent the problem of donor organ scarcity, one can resort to surgical reconstruction, whereby damaged or defective tissue at one site of the patient is replaced by healthy tissue from another part of the patient's body. These autografts include blood vessel grafts for heart bypass surgeries. The disadvantages of using autografts are their limited durability (Braunwald, Heart Disease 4th Ed., E. Braunwald (Ed.), W. B. Saunders: Philadelphia, Pa., 1992, pp. 1007-1077) and a loss of function at the donor site. In addition, reconstructive surgery often involves using the body's tissues for purposes not originally intended, which can result in long-term complications. Mechanical heart valve prostheses have proved to effectively improve patient's quality of life. However, they are also subject to mechanical failure and rejection, can induce inflammation and/or infection, and require long-term drug intervention to prevent blood-clotting. Furthermore, since these mechanical valve substitutes are nonviable, they have no potential to grow, to repair or to remodel; therefore their durability is limited, especially in growing children (Mayer Jr., Semin. Thorac. Cardiovasc. Surg. 7:130 (1995)). Since currently available treatments (with the exception of cardiac transplantation) are only palliative, new drugs and procedures for treating cardiovascular diseases, especially approaches allowing the recovery of diminished cardiac function, are highly desirable.

Tissue engineering is emerging as a significant potential alternative or complementary solution. In tissue engineering, tissue or organ failure is addressed by implanting natural, synthetic, or semi-synthetic tissue and organ mimics that are fully functional from the start or that grow into the required functionality to replace, repair, maintain and/or enhance organ/tissue function.

One of the major strategies adopted for the creation of engineered tissues is the in vitro growth of isolated cells on three-dimensional templates or scaffolds under conditions that permit the cells to develop into a functional tissue. The scaffolds, which can be fashioned from synthetic polymers or from natural materials such as collagen, temporarily provide the biomechanical support needed by the cells. As the cells grow and differentiate on the scaffold, they produce their own extracellular matrix.

The feasibility of engineered functional cardiac muscle has been demonstrated (Eschenhagen et al., FASEB J., 11:683 (1997); Freed and Vunjak-Novakovic, In Vitro Cell Dev. Biol., 33:381 (1997); Akins et al., Tissue Eng., 5:103 (1999); Bursac et al., Am. J. Physiol. Heart Circ. Physiol., 277:H433 (1999); Carrier et al., Biotechnol. Bioeng., 64:580 (1999)). Eschenhagen and co-workers (Eschenhagen et al., 1997) showed that embryonic chick cardiac myocytes cultured in collagen gels displayed characteristic physiological responses to physical and pharmacological stimuli; and Akins et al. (Akins et al., Tissue Eng., 5:103 (1999)) demonstrated that rat ventricular cardiomyocytes cultured on polystyrene microcarrier beads in bioreactors formed three-dimensional spontaneously contractile aggregates. Cultivation of neonatal rat cardiac myocytes on polyglycolic scaffolds in bioreactors has been shown to result in contractile three-dimensional tissues (Freed and Vunjak-Novakovic, 1997) with ultrastructural features of cardiac muscle.

Most tissue engineering techniques have led to cardiac muscle constructs with a number of shortcomings that limit their usefulness for both in vitro and in vivo applications. Most often, unlike native cardiac muscle that consists of fibers with a defined orientation, the cells in engineered constructs exhibit random orientation and a poor degree of differentiation. Further, the constructs often present a non-uniform spatial cell distribution with, for example, a good tissue formation at the periphery and a loose network of disoriented cells at the center of the construct. Since only a minor fraction of the three-dimensional structure consists of cardiac tissue, its usefulness as a medical implant for replacement therapy is limited.

Therefore, a need exists for functional transplantable tissue, e.g., devices and methods allowing enhanced integration and functionality of transplanted tissue in vivo.

### SUMMARY OF THE INVENTION

The present invention provides for a biological electrode (“bioelectrode”) having an electrically conducting biocompatible polymer, such as a hydrogel, and isolated donor cells capable of depolarizing and electrically coupling to other cells, e.g., cells forming a tissue, and optionally a second distinct biocompatible polymer, which polymer(s) form a scaffold. The one or more polymers in the scaffold support cell retention, survival and integration of host cells at the site of implantation of the biological electrode. In one embodiment, the invention provides a biological electrode that is biocompatible and three-dimensional, and includes donor cells and an electrically conducting biocompatible polymer, which provides a conduit for an electrical stimulus. In one embodiment, the biological electrode of the invention provides for uniform electric fields in physiologically relevant conditions, little or no failure/fatigue over time, and retention and survival of embedded donor cells, and allows embedded donor cells to electrically couple with cells in a recipient of the biological electrode, such as cardiomyocytes in a mammal. The intimate contact between the donor cells and the electrically conducting biocompatible polymer in the biological electrode dramatically lowers the energy thresholds required to induce action potentials, e.g., energy thresholds required for pacing in the cells. In one embodiment, by employing donor cells that are capable of forming gap junctions with myocardial cells, the embedded donor cells act as a biological bridge between an energy source (e.g., a pacing unit, processor and energy source) and the myocardium. In one embodiment, the donor cells capable of forming gap junctions with cardiac cells include but are not limited to cardiomyocytes, myoblasts, or stem cells, or those modified genetically to express connexins, which in turn form gap junctions. Thus, the biological electrode is useful to inhibit or treat conditions associated with reduced or aberrant electrical conduction, such as sinus node dysfunction. The present invention also provides for systems, implantable devices, and methods which employ biological electrodes having an electrically conductive biocompatible polymer and donor cells, e.g., cells with pacing potential, embedded therein.

In one embodiment, the invention provides a biological electrode having a first biocompatible polymer, an electrically conducting biocompatible polymer and mammalian donor cells. In one embodiment, the electrically conducting biocompatible polymer includes poly(pyrrole), poly(thiophene), or poly(aniline), or a combination thereof. In one embodiment, the electrically conducting biocompatible polymer is nonbiodegradable. In one embodiment, the first biocompatible polymer is configured to provide structural support for the biological electrode. In one embodiment, the first biocompatible polymer is configured to provide structural support for the biological electrode and the electrically conducting biocompatible polymer is formed on the first biocompatible polymer. In one embodiment, the first biocompatible polymer is configured to provide structural support for the biological electrode and the electrically conducting biocompatible polymer is formed on the first biocompatible polymer, wherein an electrically conductive polymeric matrix is formed by the first biocompatible polymer and the electrically conducting biocompatible polymer, and wherein donor cells are embedded in the electrically conductive polymeric matrix. In one embodiment, the donor cells in the biological electrode are capable of depolarization in response to a current, are capable of differentiating into cells that depolarize in response to a current or are capable of forming gap junctions. In one embodiment, the donor cells in the biological electrode are stem cells, e.g., mesenchymal stem cells. In one embodiment, the donor cells in the biological electrode are genetically altered.

For instance, electrically conducting biocompatible hydrogels can be used to promote depolarization of cells embedded within a three-dimensional structure, which includes the electrically conducting biocompatible polymer as well as one or more distinct biocompatible polymers. In one embodiment, the electrically conducting biocompatible polymer is nonbiodegradable. In one embodiment, the distinct biocompatible polymer is nonbiodegradable. An additional benefit associated with the biological electrodes of the invention includes an enhanced distribution of charge from a point source (current electrodes) to field sources.

In one embodiment, the invention provides a biological electrode having two distinct biocompatible polymers (a composite polymer), which may provide for better control of the polymer surface which interfaces with the host and/or three-dimensional structure. The composite polymer includes at least one polymer which provides a cell scaffold which supports growth and/or differentiation of donor cells, and an electrically conducting polymer. In one embodiment, the distinct biocompatible polymer may be a naturally-occurring polymer, e.g., chitosan, a synthetic polymer, e.g., polyethylene glycol, or combinations thereof. In one embodiment, the electrically conducting biocompatible polymer is a polypyrrole, a polythiophene or a polyaniline.

In one embodiment, an implantable device having a biological electrode of the invention is introduced (administered) to patients with sinus node dysfunction and normal AV conduction. The biological electrode includes donor cells with pacing potential, for instance, mesenchymal stem cells (MSCs), in a scaffold having an electrically conducting biocompatible polymer and a distinct biocompatible polymer. The biological electrode may be coupled to an electricity generating device and a processor, thus forming a leadless biological electrode. Current passing through the biological electrode drives cell, for instance, MSC, depolarization. In one embodiment, the cells in the biological electrode, after implant, become electrically connected with cardiomyocytes in the recipient mammal via gap junctions, which allows an action potential transmitted through the biological electrode to propagate to the rest of the heart. In various embodiments, the implantable device delivers electrical stimuli, such as cardiac pacing pulses and neurostimulation pulses, through the biological electrode to activate electrically excitable cells in the body of a patient.

The invention further provides an electrical stimulation system for delivering electrical stimulation into target tissue including target cells. The system includes a biological electrode including an electrically conductive polymeric matrix and mammalian donor cells embedded in the electrically conductive polymeric matrix; and an electrical stimulation device electrically coupled to the biological electrode, the electrical stimulation device including a stimulation output circuit adapted to deliver electrical stimulation pulses capable of depolarizing the donor cells. In one embodiment, the electrically conductive polymeric matrix includes a network of fibers of a first biocompatible polymer configured to provide structural support for the biological electrode and an electrically conducting biocompatible polymer formed on the fibers of the first biocompatible polymer. In one embodiment, the biological electrode is configured to allow action potentials to transmit from the donor cells to the target cells through gap junctions formed following placement of the biological electrode on the target tissue. In one embodiment, the electrical stimulation device in the system includes an implantable cardiac pacemaker. In one embodiment, the electrical stimulation device in the system includes an implantable neurostimulator. In one embodiment, the electrical stimulation device in the system includes an energy source including an energy harvesting device.

The present invention also provides methods in which an electrical connection between a donor cell, e.g., a transgenic donor cell, expressing a connexin and cells in a host organism, is established. In one embodiment, the present invention provides methods for inhibition or treatment of cardiac conduction disturbances which employs the biological electrode or system of the invention. Exemplary diseases amenable to treatment by such methods include, but are not limited to, complete heart block, sinus node dysfunction, reentrant arrhythmias (e.g., ventricular tachycardia), congestive heart failure, and the like. Any cardiac disease or disorder that would benefit from improved synchronized contraction is amenable to treatment. In one embodiment, the invention provides a method to treat cardiac dysfunction in a mammal, e.g., a human, by introducing the biological electrode or system of the invention to the mammal. In one embodiment, the biological electrode or system of the invention is used to treat sinus node dysfunction. In one embodiment, the biological electrode employed in the methods of the invention includes autologous, exogeneic or allogenic donor cells. In one embodiment, the biological electrode employed in the methods of the invention is connected to one or more leads.

### BRIEF DESCRIPTION OF THE FIGURES

FIG. 1 is an illustration of an embodiment of an electrical stimulation system and portions of an environment in which the electrical stimulation system operates.

FIG. 2 is an illustration of an embodiment of an electrical conductive polymeric matrix used to form a biological electrode used in the electrical stimulation system.

FIG. 3 is an illustration of an embodiment of an electrical stimulation device coupled to the biological electrode.

FIG. 4 is an illustration of another embodiment of the electrical stimulation device coupled to the biological electrode.

FIG. 5 is a block diagram illustrating an embodiment of portions of a circuit of the electrical stimulation device.

### DETAILED DESCRIPTION OF THE INVENTION

### Definitions

The term “treatment” is used herein to characterize a method that is aimed at (1) delaying or preventing the onset of a medical condition; or (2) slowing down or stopping the progression, aggravation, or deterioration of the symptoms of the condition; or (3) bringing about ameliorations of the symptoms of the condition; and/or (4) curing the condition. The treatment may be administered prior to the onset of the disease, for a prophylactic or preventive action. It may also be administered after initiation of the disease, for a therapeutic action.

The terms “individual,” “recipient” and “patient” are used herein interchangeably. They refer to a human or another mammal, that suffers from tissue deficiency, damage or loss. In one embodiment, the deficiency, damage and/or loss affect(s) a native tissue that contains electrically excitable cells and is subject to electrical stimulation in vivo.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

The term “isolated” when used in relation to a nucleic acid (polynucleotide), peptide, polypeptide, or cell, refers to a nucleic acid (polynucleotide), peptide, polypeptide, or cell that is identified and/or separated from at least one contaminant nucleic acid, polypeptide, cell type or other biological component with which it is ordinarily associated in its natural source. Isolated polynucleotide, peptide, polypeptide, or donor cells, e.g., stem cells, are present in a form or setting that is different from that in which it is found in nature. “Purified” includes when an object species is the predominant species present (e.g., it is more abundant than any other individual species in the composition), and preferably the object species comprises at least about 50 percent of all macromolecular species present. Generally, “substantially purified” includes when an object species is more than about 80 percent of all macromolecular species present in a composition, e.g., more than about 85%, about 90%, about 95%, or about 99%.

The term “peptide”, “polypeptide” and protein” are used interchangeably herein unless otherwise distinguished to refer to polymers of amino acids of any length. These terms also include proteins that are post-translationally modified through reactions that include glycosylation, acetylation and phosphorylation.

By “nucleic acid of interest” is meant any nucleic acid (e.g., DNA) which encodes a protein or other molecule which is desirable for inducing or maintaining electrical coupling between cells. In general, the nucleic acid is operatively linked to other sequences which are needed for its regulation and expression, such as a promoter and regulatory elements.

The term “biologically active” when used in particular with respect to connexin, or biological or cellular material effecting production thereof, refers to, for example, a compound having structural, regulatory, or biochemical functions of a naturally occurring connexin polypeptide, particularly with respect to facilitating the establishment of an electrochemical connection between a cell and a myocardial cell.

By “transformation”, “transduction” or “transfection” is meant a permanent or transient genetic change, preferably a permanent genetic change, induced in a cell following incorporation of new nucleic acid (e.g., DNA or RNA exogenous to the cell). Genetic change can be accomplished either by incorporation of the new nucleic acid into the genome of the host cell, or by transient or stable maintenance of the new DNA as an episomal element.

By “transformed cell”, “transfected cell” or “transduced cell” is meant a cell into which (or into an ancestor of which) has been introduced, by means of recombinant DNA techniques, a DNA molecule encoding a protein of interest.

By “construct,” “vector” or “expression cassette” is meant a recombinant nucleic acid, generally recombinant DNA, that has been generated for the purpose of the expression of a specific nucleotide sequence(s), or is to be used in the construction of other recombinant nucleotide sequences. Constructs useful in the invention are those which comprise connexin-encoding gene sequence operably linked to a promoter which will allow for the expression of the connexin protein in a transformed cell.

By “promoter” is meant a minimal sequence sufficient to direct transcription in a recombinant cell. “Promoter” is also meant to encompass those elements sufficient for promoter-dependent gene expression controllable for cell-type specific, tissue-specific or inducible by external signals or agents; such elements may be located in the 5′ or 3′ regions of the native gene (e.g., enhancer elements).

By “operably linked” or “operatively linked” is meant that a DNA sequence and a regulatory sequence(s) are connected in such a way as to permit expression when the appropriate molecules (e.g., transcriptional activator proteins) are bound to the regulatory sequence(s).

The term “implantation” or “transplantation” refers to the introduction of donor cells, e.g., cells in an implantable device, into the body of an individual.

The term “integration”, as used herein, refers to a direct functional and/or structural connection between native mammalian host (recipient) tissue and implanted donor cells.

The term “biocompatible” as used herein includes any material which upon implantation does not provoke an undesirable adverse response in a patient (e.g., an undesirable reaction other than the expected response to the trauma of implantation). When introduced into a patient, a biocompatible material is not toxic or harmful to that patient, and does not cause immunological rejection.

The term “biodegradable” as used herein refers to the ability of materials to degrade under physiological conditions to form a product that can be metabolized or excreted without damage to organs. Biodegradable materials are not necessarily hydrolytically degradable and may require enzymatic action to fully degrade. Biodegradable materials also include materials that are broken down in cells.

The term “gap junction” as used herein refers to a junction between two cells, which has pores that allow the passage of small molecules (up to 9 kD) from the cytoplasm of one cell to the cytoplasm of another cell.

The term “connexin” refers to the main protein component of a connexon, the structural subunit of a gap junction (six connexins make up one connexon).

The term “stem cell” refers to a relatively undifferentiated cell that has the capacity for sustained self-renewal, often throughout the lifetime of an animal or human, as well as the potential to give rise to differentiated progeny (i.e., to different types of specialized cells).

An “embryonic stem cell” is a stem cell derived from a group of cells called the inner cell mass, which is part of the early (4 to 5 days old) embryo called the blastocyst. Once removed from the blastocyst, the cells of the inner cell mass can be cultured into embryonic stem cells. In the laboratory, embryonic stem cells can proliferate indefinitely, a property that is not shared by adult stem cells.

An “adult stem cell” is an undifferentiated cell found in a differentiated (specialized) tissue. Adult stem cells are capable of making identical copies of themselves for the lifetime of the organism. Adult stem cells usually divide to generate progenitor or precursor cells, which then differentiate or develop into “mature” cell types that have characteristic shapes and specialized functions. Sources of adult stem cells include bone marrow, blood, the cornea and retina of the eye, brain, skeletal muscle, dental pulp, liver, skin, the lining of the gastrointestinal tract, and pancreas.

The term “pluripotent stem cell” refers to a stem cell that has the ability to give rise to types of cells that develop from the three germ layers (mesoderm, endoderm, and ectoderm) from which all the cells of the body arise.

The terms “progenitor cell” or “precursor cell” are used herein interchangeably. They refer to a cell that occurs in fetal or adult tissue and is partially specialized; it divides and gives rise to differentiated cells. Precursor cells belong to a transitory amplifying population of cells derived from stem cells. Progenitor cells do not have the capacity for sustained, undifferentiated self-renewal.

By “cardiomyocyte” is meant a cardiac contractile cell, which is a cardiac muscle cell. The cardiomyocyte cell may be isolated and cultured in vitro or be part of the myocardium of a host.

By “recombinant cell” or “transgenic cell” is meant a cell comprising nucleic acid not normally associated with the cell or in a position in the genome other than that found in nature, or a nucleic acid expressed at a level not found in a corresponding cell found in nature.

By “therapeutically effective amount” in the context of the present embodiments for treatment of cardiac conduction disturbances is meant an amount effective to decrease a symptom of cardiac conduction disturbance and/or to improve cardiac conductance (a measure of conduction).

By “overexpressing” or “overexpression” of a gene product is meant an increased level of protein expression over a normal level of protein expression for a particular cell or cell type at, for example, a particular developmental stage or stage of differentiation. In certain instances, overexpressing can be a cumulative effect of protein expression from endogenous and recombinant genes or essentially protein expression from a recombinant gene.

The terms “electrical coupling” with respect to inter-cellular junctions are intended to mean the interaction between cells which allows for intracellular communication between cells so as to provide for electrical conduction between the cells. Electrical coupling in vivo provides the basis for, and is generally accompanied by, electromechanical coupling, in which electrical excitation of cells through gap junctions in the muscle leads to muscle contraction.

By “cardiac conduction disturbance” is meant a disturbance in the normal generation and transmission of the electrical activity that initiates myocardial contraction. Cardiac arrhythmias resulting from electrical conduction disturbances can lead to life threatening ventricular tachyarrhythmias, hemodynamically compromising bradycardias, and heart block.

By “condition related to a cardiac conduction disturbance” is meant a condition, symptom or disorder associated with cardiac conduction disturbance. Examples of conditions related to cardiac conduction disturbance are irregular heart beat, fatigue, shortness of breath, and lack of synchronized heart muscle contraction.

By “connexin protein” is meant a protein from the family of homologous proteins found in connexins of gap junctions as homo- or heterohexameric arrays. Connexin proteins are the major gap junction protein involved in the electrical coupling of cells. Gap junctions regulate intercellular passage of molecules, including inorganic ions and second messengers, thus achieving electrical coupling of cells.

### General Overview

The invention provides biological electrodes having donor cells capable of pacing embedded in an electrically conducting biocompatible polymer, which optionally also has a distinct biocompatible polymer, providing a scaffold for cell retention and growth. Also provided are implantable devices having the biological electrodes which, when implanted in a host mammal, can propagate an action potential to host cells in contact with the biological electrode. The biological electrodes, devices and systems of the invention may be used to treat any disorder amenable to treatment with action potentials, e.g., patients with sinus node dysfunction and normal atrioventricular (AV) conduction, and also provide for energy harvesting, neurostimulation, and the like.

The normal cardiac electrical cycle begins with diastolic which generates an action potential and spreads from the node to depolarize the surrounding atrial tissue. The electrical impulse is then conducted through the AV node, down the His bundle to the bundle branches and distributed to the working myocardium of the ventricles through the Purkinje fiber network. For proper excitation-contraction coupling to occur, electrical conduction must be precisely timed with the corresponding mechanical contraction. Therefore, efficient cardiac contractile function is highly dependent upon the coordinated mechanical and electrical activation of the myocardial tissue. The structural integrity of the myocardium is largely achieved by the end-to-end connections between myocytes called intercalated discs. The intercalated disc consists of three main junctional complexes: adherens junctions, gap junctions and desmosomes. The adherens junction provides strong cell-cell adhesion, which is mediated by the cadherin/catenin complex via linkage to the actin cytoskeleton. The gap junction provides intercellular communication via small molecules and ions that pass through a channel generated by a family of proteins called connexins. The desmosome provides structural support through interactions of desmosomal cadherins with the intermediate filament system.

The embedded donor cells are selected as those capable of depolarizing in response to an electric current and capable of forming functional gap junctions with the cells of the recipient, e.g., cells in the myocardium of the patient. The donor cells can be from any source. In one embodiment, cardiomyocytes may be employed in the biological electrodes and devices of the invention. In another embodiment, adult bone marrow-derived human mesenchymal stem cells (hMSCs) may be employed in the biological electrodes and devices of the invention. MSCs are an autologous cell source and have been shown to express connexins and form functional gap junctions with cardiomyocytes (Valiunas et al., J. Physiol., 555:617 (2004); Beeres et al., J. Am. Coll. Cardiol., 46:1943 (2005)). In one embodiment, donor cells with pacing potential are embedded within a three-dimensional scaffold having a nonbiodegradable electrically conducting polymer. In one embodiment, the nonbiodegradable electrically conducting biocompatible polymer is a hydrogel. Hydrogels are hydrophilic, polymeric compounds that swell in aqueous environments. The use of polymers such as hydrogels may provide an improved delivery system because they can conform to the target cells or tissue. Intimate contact between donor cells and the electrically conducting biocompatible polymer dramatically lowers the energy thresholds required to induce action potentials.

In one embodiment, a biological electrode of the invention may reduce fibrosis and/or calcification, and provide for better interactions with host cells or tissue that in turn, may reduce the need for drugs and reduce collateral tissue damage associated with electricity. In one embodiment, the invention provides for a better distribution of current over a larger area (current density), thereby reducing fibrosis and lowering thresholds. In particular, the use of both conducting and nonconducting biocompatible polymers may provide a two- or three-dimensional spatially distributed electrical field, which may reduce tissue damage due to defibrillation shocks as a result of the distribution of energy over wider areas.

In one embodiment, the invention provides for the integration of a biological electrode to lower the threshold to allow for a leadless device, e.g., powered by heart motion, in which cells in the electrically conductive biocompatible polymer become a pacing center.

The biocompatible polymers may be employed to promote retention of donor cells in the biological electrode, e.g., one having an electrically conducting biocompatible polymer or one having an electrically conducting biocompatible polymer and a distinct biocompatible material, under physiological conditions for a sustained period of time, e.g., for months or years, once in the biological electrode is implanted. The polymer(s) may be biodegradable or nonbiodegradable. The electrically conducting polymer in the biological electrode may be any cell compatible, conducting polymer, e.g., polypyrrole (PPy), polythiophene, e.g., poly(3,4-ethylenedioxythiophene) (PEDOT), or polyaniline, e.g., E-PNAi. The distinct biocompatible polymer may be contacted, embedded or coated with cells or may be contacted with cells embedded in or applied to an electrically conducting biocompatible polymer. Once donor cells are embedded in or applied to an electrically conducting biocompatible polymer, including one also having a distinct biocompatible polymer, the resulting biological electrode may be coupled to an implantable medical device. Alternatively, the electrically conducting biocompatible polymer, or the electrically conducting biocompatible polymer and the distinct biocompatible polymer, may be introduced or coupled to an implantable medical device, and then donor cells contacted with, embedded in or applied to the electrically conducting biocompatible polymer or composite material.

### Electrically Conducting Polymers and Composite Polymers

Conducting polymers can promote depolarization of cells and so may promote action potentials. Surfaces formed of electrically conducting polymers are advantageous in that their properties, including surface charge, wettability, conformational and dimensional changes, can be altered reversibly by oxidation or reduction. The polymers may be applied as coatings or used to form polymeric substrates to which cells are added in, forming a scaffold.

A polymer that is intrinsically electrically conducting has an overlap of molecular orbitals to allow the formation of delocalized molecular wave function. Besides this, molecular orbitals must be partially filled so that there is a free movement of electrons through the lattice (Bloor et al., IEEE-Proceedings, 130:225 (1983)). Conducting polymers contain a π-electron backbone responsible for their unusual conductivity, low energy optical transitions, low ionization potential and high electron affinity. This extended π-conjugated system of the conducting polymers has single and double bonds alternating along the polymer chain.

Thus, electrically conducting polymers represent a class of materials whose electrical and optical properties can be controllably varied over an extremely wide range, oftentimes in a completely reversible manner. This may be accomplished either by chemical or electrochemical oxidation of the 7′-system of the polymer backbone or, in some cases, by direct protonation of the polymer backbone. It is possible to systematically vary the electrical conductivity of these materials from the insulating state to the conducting state. The polymer supports positive charges that are delocalized over relatively short segments of the backbone.

Representative electrically conducting polymers include polyacetylene, polyaniline, polypyrrole, polythiophene, poly(phenylenesulfide), and poly(phenylenevinylene). The terms “polyanilines”, “polypyrroles” and “polythiophenes,” includes polyaniline, polypyrrole, polythiophenes, and derivatives thereof, which can be made using methods available in the art. Derivatives which can be used include substituted polyanilines, polypyrroles and polythiophenes, such as N-substituted polypyrroles. Additionally, 3-substituted polyanilines, polypyrroles, and polythiophenes can be used, such as 3-alkyl substituted derivatives. Pyrrole can be either chemically or electrochemically polymerized to form polypyrrole. Methods of preparing conductive polymers are disclosed, e.g., in U.S. Pat. No. 6,095,148. Polymers can be deposited as coatings onto substrates or polymerized to form objects. Polymers can be applied as a single layer coating of a single polymer or as a multilayered film to alter the properties of the applied polymers.

A number of classes of conductive polymers have been shown to be useful in biological systems. These include poly(acetylene)s, poly(pyrrole)s, poly(thiophene)s and poly(aniline)s, e.g., polypyrrole (PPy) and PEDOT. PPy and PEDOT have excellent electrical properties and may be compatible with cardiac applications. For instance, Bidez et al. (J. Biomat. Sci. Polymer Ed., 17:199 (2006)) showed that the electrically conducting polymer polyaniline (E-PNAi) was compatible with cardiac myoblast adhesion and proliferation, and was conductive in aqueous physiologic environments.

Electrically conducting biocompatible polymers, e.g., electrically conducting biocompatible hydrogels, useful in the biological electrode of the invention are selected to function under electrical parameters (conduction, impedance, etc.) in aqueous conditions, so as to deliver uniform electric fields throughout the polymer in physiologically relevant salt conditions. Electrically conducting biocompatible polymers are also selected to provide for donor cell survival and retention, and to allow for coupling of donor cells to host recipient cells, e.g., cardiomyocytes. Pacing may be delivered in vitro to biological electrodes having biocompatible polymers with donor cells in proximity to cardiomyocytes, to assess whether the donor cells modify the intrinsic cell pacing rhythms of co-cultured cardiomyocytes. The identification of a physical connection between two cells can be readily determined by those skilled in the art. For instance, the presence of gap junctions can be evaluated by microinjecting cells with a gap junction permeable dye, which is transferred from one cell to another when functional gap junctions are present.

Various methods are available for the synthesis of conducting polymers. However, the most widely used technique is the oxidative coupling involving the oxidation of monomers to form a cation radical followed by coupling to form di-cations and the repetition leads to the polymer. Electrochemical synthesis is one general method for preparing electrically conducting polymers. Electrochemical polymerization of conducting polymers is generally employed using constant current or galvanostatic; constant potential or potentiostatic; or potential scanning/cycling or sweeping methods.

Electrically conducting polymers may be combined with another distinct biocompatible polymer to form a cell scaffold (a composite polymer). Moreover, the properties of polymers can be modified using cross-linking agents. The distinct polymer may be formed of any of a wide range polymers including naturally occurring polymers, synthetic polymers, or a combination thereof, and including biodegradable polymers or nonbiodegradable polymers. For instance, a naturally occurring biodegradable polymer may be modified to provide for a nonbiodegradable polymer derived from the naturally occurring polymer. In one embodiment, conductive polymers can be blended with biocompatible biodegradable polymers or nonbiodegradable polymers, such as a poly(lactic acid) or poly(lactic-co-glycolic acid) (“PLGA”). In one embodiment, the biological electrodes include one or more polymers permitting cells to migrate into, attach and proliferate within the polymers. In one embodiment, cells and subunits of the polymers are combined prior to polymerization, and after polymerization, provides for cells encapsulated within a three dimensional polymer structure. In one embodiment, one of the biocompatible materials includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Hydrogels

Hydrogels are hydrophilic polymeric networks, with chemical or physical crosslinks, that are capable of swell and can retain a large amount of water. Many hydrogels exhibit biocompatibility, and cause minimal inflammatory responses, thrombosis, and tissue damage (Graham, Med. Device Tech., 9:18 (1998), Graham, Med. Device Tech., 9:22 (1998)). In addition, hydrogels have high permeability for oxygen, nutrients, and other water-soluble metabolites. Some types of hydrogels can be photopolymerized in vivo and in vitro in the presence of photoinitiators using visible or ultraviolet (UV) light. Photopolymerization is used to convert a liquid monomer or macromer to a hydrogel by free radical polymerization in a fast and controllable manner under ambient or physiological conditions.

Visible or UV light can interact with light-sensitive compounds called photoinitiators to create free radicals that can initiate polymerization to form crosslinked hydrogels (Scranton et al., In: Photopolymerization Fundamentals and Applications, New Orleans, ACS Publishers (1996)). Photopolymerization has several advantages over conventional polymerization techniques. These include spatial and temporal control over polymerization, fast curing rates (less than a second to a few minutes) at room or physiological temperatures, and minimal heat production.

Three major classes of photoinitiation, depending on the mechanism involved in photolysis, include radical photopolymerization through photocleavage, hydrogen abstraction, and cationic photopolymerization. The radical photopolymerization by photocleavage involves photoinitiators that undergo cleavage at C—C, C—Cl, C—O, or C—S bonds to form radicals when exposed to light. These photoinitiators include aromatic carbonyl compounds such as benzoin derivatives, benziketals, acetophenone derivatives, and hydroxyalkylphenones. Acetophenone derivatives, such as 2,2-dimethoxy-2-pheyl acetophenone, have been used as photoinitiators to form hydrogels from acrylated polyethylene glycol (PEG) derivatives. Upon UV irradiation, photoinitiators such as aromatic ketones (i.e., benzophenone and thioxanthone) undergo hydrogen abstraction from an H-donor molecule to generate a ketyl radical and a donor radical. The photoinitiator isopropyl thioxanthone has been shown to be cytocompatible (Bryant et al., J. Biomater. Sci. Polymer. Ed., 11:439 (2000)).

Photopolymerizable hydrogels may be formed from macromolecular hydrogel precursors. These are water-soluble polymers with two or more reactive groups. Examples of photopolymerizable macromers include PEG acrylate derivatives, polyvinyl alcohol (PVA) derivatives, and modified polysaccharides such as hyaluronic acid derivatives and dextran methacrylate.

The hydrogel material may provide immunoisolation yet allows facile diffusion of oxygen, nutrients, and metabolic products.

Because the mechanical properties of many hydrogels can be tailored to match those of many soft tissues, those polymeric materials alone may be employed in the biological electrodes of the invention.

### Biocompatible Polymers for the Biological Electrode

In one embodiment, a polymer(s) useful in the composite polymers may be made of any suitable material, e.g., which is nonbiodegradable and biocompatible, e.g., agarose, polyvinyl alcohol, e.g., cross-linked polyvinyl alcohol, polyacrylates, polyamides, and polyurethane, and including a dialysis membrane, nylon, or polysulfoxy, or cellulose, which allows the transport of low molecular weight substances inward and outward, permitting cell survival and function, and may prevent entry and exit of large molecules.

Suitable biocompatible materials for the polymers include but are not limited to polyacetic or polyglycolic acid and derivatives thereof, polyorthoesters, polyesters, polyurethanes, polyamino acids such as polylysine, lactic/glycolic acid copolymers, polyanhydrides and ion exchange resins such as sulfonated polytetrafluorethylene, polydimethyl siloxanes (silicone rubber) or combinations thereof.

In one embodiment, the distinct polymer is formed from natural proteins or materials which may be crosslinked using a crosslinking agent such as 1-ethyl-3-(3 dimethylamino-propyl)carbodiimide hydrochloride. Such natural materials include albumin, collagen, fibrin, alginate, extracellular matrix (ECM), e.g., xenogeneic ECM, hyaluronan, chitosan, gelatin, keratin, potato starch hydrolyzed for use in electrophoresis, and agar-agar (agarose), or other “isolated materials”. An “isolated” material has been separated from at least one contaminant structure with which it is normally associated in its natural state such as in an organism or in an in vitro cultured cell population.

The biocompatible materials include synthetic polymers in the form of hydrogels or other porous materials, e.g., permeable configurations or morphologies, such as polyvinyl alcohol, polyvinylpyrrolidone and polyacrylamide, polyethylene oxide, poly(2-hydroxyethyl methacrylate); natural polymers such as gums and starches; synthetic elastomers such as silicone rubber, polyurethane rubber; and natural rubbers, and include poly[α(4-aminobutyl)]-1-glycolic acid, polyethylene oxide (Roy et al., Mol. Ther., 7:401 (2003)), polyorthoesters (Heller et al., Adv. Drug Delivery Rev., 54:1015 (2002)), silk-elastin-like polymers (Megeld et al., Pharma. Res., 19:954 (2002)), alginate (Wee et al., Adv. Drug Deliv. Rev., 31:267 (1998)), EVAc (poly(ethylene-co-vinyl acetate), microspheres such as poly(D, L-lactide-co-glycolide) copolymer and poly (L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, an agarose hydrogel, or a lipid microtubule-hydrogel.

In one embodiment, donor cells are embedded in or applied to a biocompatible material, e.g., a nonionic or ionic nonbiodegradable material, including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material for the distinct polymer is derived from isolated ECM. ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like. The preparation and use of isolated ECM in vivo is described in co-pending, commonly assigned U.S. patent application Ser. No. 11/017,237, entitled “USE OF EXTRACELLULAR MATRIX AND ELECTRICAL THERAPY,” filed on Dec. 20, 2004, which is hereby incorporated by reference in its entirety.

In one embodiment, ECM is isolated from the small intestine. Intestinal submucosal tissue for use in the invention typically comprises the tunica submucosa delaminated from both the tunica muscularis and at least the luminal portions of the tunica mucosa. In one embodiment, the submucosal tissue comprises the tunica submucosa and basilar portions of the tunica mucosa including the lamina muscularis mucosa and the stratum compactum. The preparation of submucosal tissue is described in U.S. Pat. No. 4,902,508 and Bell, In: Tissue Engineering: Current Perspectives, Cambridge, Mass., Burkhauser Publishers, pp. 179-189 (1993), the disclosures of which are expressly incorporated herein by reference. For example, a segment of vertebrate intestine, preferably harvested from porcine, ovine or bovine species, or other warm blooded vertebrates, is rinsed free of contents, then split longitudinally to form a sheet and delaminated. In particular, the superficial layers of the tunica mucosa are removed by mechanical delamination. The tissue is then turned to the opposite side and the tunica muscularis externa and tunica serosa are mechanically removed leaving the tunica submucosa and the basilar layers of the tunica mucosa. The remaining tissue represents isolated ECM and may include a small number of intact cells.

In one embodiment, ECM is isolated from the urinary bladder. The wall of the urinary bladder is composed of the following layers: the mucosa (including a transitional epithelium layer and the tunica propria), a submucosa layer, up to three layers of muscle and the adventitia (a loose connective tissue layer)—listed in cross-section from luminal to abluminal sides. Urinary bladder submucosa may be prepared from bladder tissue harvested from animals raised for meat production, including, for example, porcine, ovine or bovine species or other warm-blooded vertebrates. For example, the urinary bladder is harvested and thoroughly rinsed in tap water to remove its contents. The bladder is split open through the apex and bisected to yield roughly equal-sized halves that are prepared separately. The luminal side of the bladder is placed face down and the external muscle layers, i.e., muscularis externa (smooth muscle cell layers and serosa), are removed by mechanical delamination. The transitional epithelium of the urinary bladder is removed by either mechanical or ionic methods (e.g., 1.0 N NaCl treatment) leaving behind tissue corresponding to isolated ECM, e.g., approximately a 50 μM to 80 μM thick sheet of ECM that originally resides between the transitional epithelium and the smooth muscle layers of the urinary bladder, i.e., the submucosa and basement membrane of the transitional epithelium.

In another embodiment, ECM from bladder wall segments or small intestine is prepared using a modification to the technique in Meezan et al. (Life Sci., 17:1721 (1975)). The method in Meezan et al. includes placing tissue fractions in a large volume (100:1) of distilled water containing 0.1% sodium azide and magnetically stirring the mixture for 1-2 hours in order to lyse the cells and release the intracellular contents. The lysed tissue suspension is then centrifuged to yield a firm pellet, and the supernatant discarded. The pellet is suspended in 40 ml of 1M NaCl and 2000 Kunitz units of DNAase (Sigma, Deoxyribonuclease 1) are added and the suspension stirred for 1-2 hours. The mixture is again centrifuged to bring down a firm pellet and the supernatant discarded. The pellet is then suspended in 40 ml of 4% sodium deoxycholate containing 0.1% sodium azide and stirred for 2-4 hours at room temperature. The mixture is centrifuged, the supernatant discarded, and the pellet either washed several times with water by centrifugation and resuspension, or by extensive irrigation on a 44 micron nylon sieve (Kressilk Products, Inc., Monterey Park, Calif.). In the modified method, the time of incubation with sodium deoxycholate and sodium azide is increased and additional washing procedures incorporated. Accordingly, first, the mucosa is scraped off mechanically. Afterwards all cell structures of the remaining tissue are eliminated chemically and enzymatically leaving the acellularized muscularis layer. This is achieved with subsequent exposure to a hypoosmolar and hyperosmolar solutions of crystalloids. In addition, a final treatment with sodium deoxycholate destroys remaining cell structures. After following washing procedures, the resulting material, which provides cross-linked fibres of the submucosa with the remaining muscularis collagen-elastin framework, can be stored in PBS solution, e.g., with antibiotics at 4° C. for a few months.

Isolated ECM can be cut, rolled, or folded.

Fluidized forms of submucosal tissue are prepared by comminuting submucosa tissue by tearing, cutting, grinding, or shearing the harvested submucosal tissue. Thus, pieces of submucosal tissue can be comminuted by shearing in a high speed blender, or by grinding the submucosa in a frozen or freeze-dried state, to produce a powder that can thereafter be hydrated with water or buffered saline to form a submucosal fluid of liquid, gel or paste-like consistency.

The comminuted submucosa formulation can further be treated with an enzymatic composition to provide a homogenous solution of partially solubilized submucosa. The enzymatic composition may comprise one or more enzymes that are capable of breaking the covalent bonds of the structural components of the submucosal tissue. For example, the comminuted submucosal tissue can be treated with a collagenase, glycosaminoglycanase, or a protease, such as trypsin or pepsin at an acidic pH, for a period of time sufficient to solubilize all or a portion of the submucosal tissue protein components. After treating the comminuted submucosa formulation with the enzymatic composition, the tissue is optionally filtered to provide a homogenous solution. The viscosity of fluidized submucosa for use in accordance with this invention can be manipulated by controlling the concentration of the submucosa component and the degree of hydration. The viscosity can be adjusted to a range of about 2 to about 300,000 cps at 25° C. Higher viscosity formulations, for example, gels, can be prepared from the submucosa digest solutions by adjusting the pH of such solutions to about 6.0 to about 7.0.

The present invention also contemplates the use of powder forms of submucosal tissues. In one embodiment, a powder form of submucosal tissue is prepared by pulverizing intestinal submucosa tissue under liquid nitrogen to produce particles ranging in size from 0.01 to 1 mm² in their largest dimension. The particulate composition is then lyophilized overnight, pulverized again and optionally sterilized to form a substantially anhydrous particulate composite. Alternatively, a powder form of submucosal tissue can be formed from fluidized submucosal tissue by drying the suspensions or solutions of comminuted submucosal tissue.

Submucosal tissue may be “conditioned” to alter the viscoelastic properties of the submucosal tissue. Submucosal tissue is conditioned by stretching, chemically treating, enzymatically treating or exposing the tissue to other environmental factors. The conditioning of submucosal tissue is described in U.S. Pat. No. 5,275,826, the disclosure of which is expressly incorporated herein by reference. In accordance with one embodiment, vertebrate derived submucosal tissues are conditioned to a strain of no more than about 20%.

In one embodiment, the submucosal tissue is conditioned by stretching the tissue longitudinally. One method of “conditioning” the tissue by stretching involves application of a given load to the submucosa for three to five cycles. Each cycle consists of applying a load to the tissue for five seconds, followed by a ten second relaxation phase. Three to five cycles produces a stretch-conditioned material. For example, submucosal tissue can be conditioned by suspending a weight from the tissue, for a period of time sufficient to allow about 10 to 20% or more elongation of the tissue segment. Optionally, the material can be preconditioned by stretching in the lateral dimension.

In one embodiment the submucosal tissue is stretched using 50% of the predicted ultimate load. The “ultimate load” is the maximum load that can be applied to the submucosal tissue without resulting in failure of the tissue (i.e., the break point of the tissue). Ultimate load can be predicted for a given strip of submucosal tissue based on the source and thickness of the material. Accordingly, one method of “conditioning” the tissue by stretching involves application of 50% of the predicted ultimate load to the submucosa for three to ten cycles. Each cycle consists of applying a load to the material for five seconds, followed by a ten second relaxation phase. The resulting conditioned submucosal tissue has a strain of less than 30%, more typically a strain from about 20% to about 28%. In one embodiment, conditioned the submucosal tissue has a strain of no more than 20%. The term strain as used herein refers to the maximum amount of tissue elongation before failure of the tissue, when the tissue is stretched under an applied load. Strain is expressed as a percentage of the length of the tissue before loading.

Typically the conditioned submucosal tissue is immobilized by clamping, suturing, stapling, gluing (or other tissue immobilizing techniques) the tissue to the support, wherein the tissue is held at its preconditioned length in at least one dimension. In one embodiment, delaminated submucosa is conditioned to have a width and length longer than the original delaminated tissue and the conditioned length and width of the tissue is maintained by immobilizing the submucosa on a support. The support-held conditioned submucosal tissue can be sterilized before or after being packaged.

The isolated ECM is decellularized, and optionally sterilized, prior to storage and/or use. In one embodiment, isolated ECM has a thickness of about 50 to 250 micrometers, e.g., 100 to 200 micrometers, and is >98% acellular. Numerous methods may be used to decellularize isolated ECM (see, for example, Courtman et al., J. Biomed. Materi. Res., 18:655 (1994); Curtil et al., Cryobiology, 34:13 (1997); Livesey et al., Workshop on Prosthetic Heart Valves, Georgia Inst. Tech. (1998); Bader et al., Eur. J. Cardiothorac. Surg., 14:279 (1998)). For instance, treatment of isolated ECM with dilute (0.1%) peracetic acid and rinsing with buffered saline (pH 7.0 to 7.4) and deionized water renders the material acellular with a neutral pH. Alternatively, isolated ECM is thoroughly rinsed under running water to lyse the remaining resident cells, disinfected using 0.1% peracetic acid in ethanol, and rinsed in phosphate buffered saline (PBS, pH=7.4) and distilled water to return its pH to approximately 7.0. Decellularization may be ascertained by hematoxylin-eosin staining.

Isolated and decellularized ECM contains a mixture of structural and functional molecules such as collagen type I, III, IV, V, VI; proteoglycans; glycoproteins; glycosaminoglycans, and may contain growth factors, in their native 3-dimensional microarchitecture, including proteins that influence cell attachment, gene expression patterns, and the differentiation of cells. Isolated ECM is optionally sterilized and may be stored in a hydrated or dehydrated state.

Isolated ECM may be sterilized using conventional sterilization techniques including tanning with glutaraldehyde, formaldehyde tanning at acidic pH, ethylene oxide treatment, propylene oxide treatment, gas plasma sterilization, gamma radiation, electric beam radiation and peracetic acid sterilization. Sterilization techniques which do not adversely affect the mechanical strength, structure, and biotropic properties of the isolated ECM are preferred. For instance, strong gamma radiation may cause loss of strength of sheets of submucosal tissue. Preferred sterilization techniques include exposing isolated ECM to peracetic acid, low dose gamma irradiation, e.g., 1-4 mRads gamma irradiation or more preferably 1-2.5 mRads of gamma irradiation, or gas plasma sterilization. In one embodiment, peracetic acid treatment is typically conducted at a pH of about 2 to about 5 in an aqueous ethanolic solution (about 2 to about 10% ethanol by volume) at a peracid concentration of about 0.03 to about 0.5% by volume. After isolated ECM is sterilized, it may be wrapped in a porous plastic wrap or foil and sterilized again, e.g., using electron beam or gamma irradiation sterilization techniques. Isolated ECM for implantation is generally subjected to two or more sterilization processes. Terminal sterilization, e.g., with 2.5 mRad (10 kGy) gamma irradiation results in a sterile, pyrogen-free biomaterial. Isolated ECM or isolated, decellularized ECM may then be stored, e.g., at 4° C., until use. Lyophilized or air dried ECM can be rehydrated and used in accordance with this invention without significant loss of its properties. Decellularized and/or sterilized isolated ECM is substantially nonimmunogenic and has high tensile strength. Isolated ECM may, upon implantation, undergo remodeling (resorption and replacement with autogenous differentiated tissue), serve as a rapidly vascularized matrix for support and growth of new tissue, and assume the characterizing features of the tissue(s) with which it is associated at the site of implantation, which may include functional tissue.

In some embodiments, isolated ECM may be subjected to chemical and non-chemical means of cross-linking to modify the physical, mechanical or immunogenic properties of naturally derived ECM (Bellamkondra et al., J. Biomed. Mater. Res., 29:633 (1995)). Chemical cross-linking methods generally involve aldehyde or carbodiimide. Photochemical means of protein cross-linking may also be employed (Bouhadir et al., Ann. NY Acad. Sci., 842:188 (1998)). Cross-linking generally results in a relatively inert bioscaffold material which may induce a fibrous connective tissue response by the host to the scaffold material, inhibit scaffold degradation, and/or inhibit cellular infiltration into the scaffold. ECM scaffolds that are not cross-linked tend to be rapidly resorbed in contrast nonresorbable cross-linked materials or synthetic scaffolds such as Dacron or polytetrafluoroethylene (Bell, Tissue Engin., 1:163 (1995); Bell, In: Tissue Engineering: Current Perspectives, Burhauser Pub. pp. 179-189 (1993); Badylak et al., Tissue Engineering, 4:379 (1998); Gleeson et al., J. Urol., 148:1377 (1992)).

Seeding of biocompatible materials with cells can be performed prior to and/or at the time of implantation. In one embodiment, seeding of biocompatible materials can be performed in a static two-dimensional chamber system or a three-dimensional rotating bioreactor. For instance, wet isolated ECM (2×3 cm in size) or tubular segments to be seeded are placed on the bottom of a chamber and covered with a liquid medium such as an aqueous medium, e.g., a cell culture medium, or perfused with such medium, for instance, over a period of up to 6 weeks. For instance, 1×10⁶ to 1×10¹² cells may be added to a biocompatible polymer. Nevertheless, the number of cells within a polymer may vary, depending on the volume of the polymer, cell type employed, and target area. Cells may attach to polymers suitable as scaffolds, e.g., to isolated ECM via several attachment proteins present within scaffolds, including type I collagen, type IV collagen, and fibronectin (Hodde et al., Tissue Engineering, 8:225 (2002)).

The above examples are provided for reference only, and the range of suitable materials should not be construed as limited to those materials listed above.

### Donor Cells

Sources for donor cells include but are not limited to bone marrow-derived cells, e.g., mesenchymal cells and stromal cells, smooth muscle cells, fibroblasts, SP cells, pluripotent cells or totipotent cells, e.g., teratoma cells, hematopoietic stem cells, for instance, cells from cord blood and isolated CD34⁺ cells, adult stem cells, e.g., multipotent adult progenitor cells (MAPCs), embyronic stem cells, skeletal muscle derived cells, for instance, skeletal muscle cells and skeletal myoblasts, cardiac derived cells, myocytes, e.g., ventricular myocytes, atrial myocytes, SA nodal myocytes, AV nodal myocytes, and Purkinje cells. The term “donor cell” includes embryonic, fetal, pediatric, or adult cells or tissues, including but not limited to, stem cells and precursors (progenitor) cells. Thus, donor cells of the invention can be myocardial cells, bone marrow cells, hematopoietic cells, lymphocytes, leukocytes, granulocytes, monocytes, macrophages, neural cells, mesenchymal stem cells, and combinations thereof, or cells capable of differentiating into those cells. In one embodiment, the donor cells are autologous cells, however, non-autologous cells, e.g., xenogeneic or allogeneic cells, may be employed. Donor cells may be isolated from cardiac tissue, skeletal muscle tissue, bone marrow, e.g., MSCs, or umbilical cord blood. These or similar cell populations may be capable of differentiation into electrically excitable cells, e.g., cells with gap junctions such as cardiac cells. Methods of culturing cells and/or methods of inducing differentiation of cells are known to the art. For example, methods to induce differentiation of ES cells, bone marrow cells, or hematopoietic stem cells to cardiac cells, are described in U.S. patent application Ser. No. 10/722,115, entitled “METHOD AND APPARATUS FOR CELL AND ELECTRICAL THERAPY OF LIVING TISSUE”.

Stem cells may be isolated from any source known in the art and includes, but is not limited to, e.g., peripheral blood stem cells (PBSC), stem cells isolated from bone marrow; stem cells isolated from adipose tissue; mesenchymal stem cells, embryonic stem cells, CD34⁺ cells, CD34⁻ cells, CD45⁺ cells, or combinations thereof).

In one embodiment, cells for use in the present invention are cardiac or neuronal cells, e.g., neuronal stem cells. Cardiac cells and neuronal cells may be obtained from various sources such as donated organs or live donors. Heart tissue or cells and neuronal tissue or cells from either a donated organ or a live donor may be further cultured prior to use. Methods for culturing cardiac tissue or cells are well known and can be found in, for example, Rust et al. (Mol. Cell. Biochem., 181:143 (1998)). Methods for culturing neuronal tissue or cells are also well known and may be found in, for example, Bamea et al. (Res. Protoc., 4:156 (1999)). The donor tissue or cells and biosensor recipient should be as closely phylogenetically related as possible. For example, when the biosensor recipient is a human, cardiac tissue or cells and/or neuronal cells from a human or pig may be used. Preferably, human cells are implanted in a human. Thus, a recipient may serve as a cell donor. Alternatively, cardiac cells or neuronal cells from a donor other than the recipient may be used. When cardiac or neuronal cells from a donor other than the recipient is used, the tissue or cells and the individual recipient are preferably HLA typed and matched.

The cells may be stem cell-derived cardiac myocytes. In this embodiment, stem cells are used to culture cardiac myocytes. Stem cells may be obtained from various sources such as, e.g., bone marrow, peripheral blood, organs, or tissue, including fat or umbilical cord blood, as well as any combination of these sources.

The donor cells can optionally be expanded in vitro to provide an expanded population of donor cells and/or to provide a two- or three-dimensional structure, optionally in combination with a biocompatible material, e.g., matrix. For instance, donor cells may be treated in vitro by subjecting them to mechanical, electrical, or biological conditioning, or any combination thereof, as described in U.S. patent application Ser. No. 10/722,115, entitled “METHOD AND APPARATUS FOR CELL AND ELECTRICAL THERAPY OF LIVING TISSUE”, which is incorporated by reference herein. The conditioning may include continuous or intermittent exposure to exogenous stimuli. Mechanical conditioning includes subjecting donor cells to a mechanical stress that simulates the mechanical forces applied upon cardiac muscle cells in the myocardium due to the cyclical changes in heart volume and blood pressure. Electrical conditioning includes subjecting donor cells to electrical conditions that simulate the electrical conditions in the myocardium which result in contraction of the heart. Biological conditioning includes subjecting donor cells to exogenous agents, e.g., differentiation factors, growth factors, angiogenic proteins, survival factors, and cytokines, as well as to expression cassettes (transgenes) optionally in addition to the expression cassette encoding a gene product such as a protein. In one embodiment, donor cells include myoblasts, fetal cardiomyocytes, stem cells such as adult bone marrow stem cells, fibroblasts, or other suitable cells that provide sufficient gap junctions or other cell types modified to express sufficient connexins.

In one embodiment, a distinct biocompatible polymer is mixed with or added to an electrically conducting biocompatible polymer to provide a composite polymer, and donor cells are added to the composite polymer, to form a biological electrode. In one embodiment, donor cells are contacted or mixed with an electrically conducting biocompatible polymer, which is then mixed with or added to a distinct biocompatible polymer, to form a biological electrode. In another embodiment, donor cells are mixed with or added to a distinct biocompatible polymer, which is then mixed with an electrically conducting biocompatible polymer, to form a biological electrode. In yet another embodiment, donor cells and a distinct biocompatible polymer, e.g., one suitable as a cell scaffold, and an electrically conducting biocompatible polymer are mixed to form a biological electrode. In another embodiment, monomers or other subunits for a distinct biocompatible polymer and for an electrically conducting biocompatible polymer are mixed with donor cells to form a biological electrode.

### Exemplary Disorders and Gap Junctions Useful in Treating Those Disorders

Gap junctions are plaques of multiple intercellular channels that connect the cytoplasm of adjacent cells. An individual channel is created by stable, noncovalent interactions of two hemichannels, referred to as connexons. Each connexon is composed of six connexin proteins (see Tables 1 and 2). A major role of gap junctions in the myocardium is to enable rapid and coordinated electrical excitation, a prerequisite for normal rhythmic cardiac function. In the mammalian heart, gap junction channels are mainly composed of three different types of connexin protein, Cx43, Cx40 and Cx45, whose expression is subject to spatio-temporal and species-specific regulation. Cx43 is the main constituent of cardiac gap junctions and in the rodent it is expressed in all atrial and ventricular myocytes. In the rodent heart Cx40 is expressed in atrial myocytes and in the AV conducting system, while Cx45 is expressed at significant levels only in the conductive system and the epicardial coronary arteries. The co-localization of different connexin proteins in gap junction plaques, observed immunohistochemically, probably reflects the formation of heterotypic or heteromeric gap junction channels (Honjo et al., Cardio. Res., 53:89 (2002); Kanter et al., Circ. Res., 73:344 (1963); Yeh et al., Circ. Res., 83:1248 (1998); Saffitz et al., Circ. Res., 94:585 (2004)). Cardiac myocytes actively regulate the level of coupling they have to neighboring cells by multiple mechanisms, which include alterations in connexin expression, regulation of trafficking and turnover, and modulation of channel properties.

TABLE 1

| Mouse connexins | Human connexins |
|---|---|
| Cx23 | Cx23 |
| | Cx25 |
| Cx26 | Cx26 |
| Cx29 | Cx30.2 |
| Cx30 | Cx30 |
| Cx30.2 | Cx31.9 |
| Cx30.3 | Cx30.3 |
| Cx31 | Cx31 |
| Cx31.1 | Cx31.1 |
| Cx32 | Cx32 |
| Cx33 | |
| Cx36 | Cx36 |
| Cx37 | Cx37 |
| Cx39 | Cx40.1 |
| Cx40 | Cx40 |
| Cx43 | Cx43 |
| Cx45 | Cx45 |
| Cx46 | Cx46 |
| Cx47 | Cx47 |
| Cx50 | Cx50 |
| | Cx59 |
| Cx57 | Cx62 |

TABLE 2

| | Representative | |
|---|---|---|
| Mouse connexins | tissue/organ | Representative cell type |
| Cx23 | — | — |
| Cx26 | Liver, skin | Hepatocytes, |
| | | keratinocytes |
| Cx29 | Brain | Oligodendrocytes |
| Cx30 | Skin | Keratinocytes |
| Cx30.2 | Testis | Smooth-muscle |
| | | cells |
| Cx30.3 | Skin | Keratinocytes |
| Cx31 | Skin | Keratinocytes |
| Cx31.1 | Skin | Keratinocytes |
| Cx32 | Liver, nervous | Hepatocytes, |
| | | Schwann cells |
| Cx33 | Testes | Sertoli cells |
| Cx36 | Retina, nervous | Neurons |
| Cx37 | Blood vessels | Endothial cells |
| Cx39 | Developing muscle | Myocytes |
| Cx40 | Heart, skin | Cardiamyocytes, |
| | | keratinocytes |
| Cx43 | Heart, skin | Cardiamyocytes, |
| | | keratinocytes |
| Cx45 | Heart, skin | Cardiamyocytes, |
| | | keratinocytes |
| Cx46 | Lens | Lens fibre cells |
| Cx47 | Nervous | Oligodendrocytes |
| Cx50 | Lens | Lens fibre cells |
| Cx57 | Retina | Horizontal cells |

In the diseased heart, Cx43 is often down-regulated, redistributed and preferentially lost from the intercalated disc (Peters et al., Circ., 88:864 (1993); Dupon et al., J. Mol. Cell. Cardiol., 33:359 (2001); Smith et al., Am. J. Path., 139:801 (1991)). Disturbances in the distribution of gap junctions and reduced levels of Cx43 occur not only in association with established infarct scar tissue in the human heart, but have been shown in experimental animals to be initiated rapidly after ventricular ischemia and infarction (Matsushita et al., Circ. Res., 85:1045 (1999); Deleau et al., Can. J. Physiol. Pharma, 79:371 (2001)). Alterations in connexin expression and spatial remodeling of gap junctions in regions bordering healing infarcts have been implicated in the development of slow, heterogeneous conduction and conduction block critical in reentrant arrhythmogenesis (Peters et al., Circ., 95:988 (1997)). More widespread spectacular disordered arrangements of ventricular Cx43 gap junctions are an inevitable consequence of the haphazard myocyte organization characteristic of human hypertrophic cardiomyopathy, the most common cause of SCD due to arrhythmia (Sepp et al., Heart, 76:412 (1991)).

In one embodiment, donor cells are genetically altered (transgenic donor cells) to include an expression cassette encoding a connexin, to provide for transgenic donor cells with enhanced gap junction formation. The transgenic donor cells can optionally be genetically modified to express other proteins, such as a N-cadherin protein. The expression cassette may include a constitutive promoter or a regulatable, e.g., an inducible promoter, operably linked to a connexin. The use of such transgenic donor cells, or nontransgenic donor cells that are capable of expressing gap junctions, may be useful to inhibit or treat diseases shown in Table 3.

TABLE 3

| Gene mutation | Species | Disease phenotype |
|---|---|---|
| Connexin40 | Mouse | AV block, inducible atrial |
| | | tachyarrhythmia |
| Connexin40 | Human | Atrial standstill, co-inherited with |
| (−44G→ A; +71 | | cardiac sodium channel gene |
| A→ G) | | (SCN5A) Asp1275 Asn mutation |
| Connexin43 | Mouse | Spontaneous ventricular tachycardia, |
| | | slow conduction velocity, SCD |
| Connexin43 | Mouse | Oculodentodigital dysplasia (syndactyly, |
| (Gly60Ser) | | enamel hypoplaisa, craniofacial |
| | | anomolies and cardiac dysfunction). |
| | | Mild first degree AV block, irregular |
| | | sinus with AV dissociation and |
| | | junctional escape, bradycardia |

In one embodiment, the DNA construct contains a promoter to facilitate expression of the DNA of interest within a mammalian cell. The promoter may be a strong promoter that functions in mammalian cells, such as a promoter from cytomegalovirus (CMV), mouse mammary tumor virus (MMTV), Rous sarcoma virus (RSV), lentivirus or adenovirus. Alternatively, the promoter used may be a strong general eukaryotic promoter such as the actin gene promoter. In one embodiment, the promoter used may be a tissue-specific promoter. For example, the promoter used in the construct may be a cardiac cell specific promoter, a myoblast specific promoter or an adult skeletal muscle cell specific promoter.

In another embodiment, the promoter is a regulatable promoter (e.g., inducible promoter), such as a tetracycline-regulated promoter, expression from which can be regulated by exposure to an exogenous substance (e.g., tetracycline). Another example of regulated promoter system useful in the present invention is the lac operator repressor system.

Expression in the modified cells can be detected by such techniques as western blotting, utilizing antibodies specific for the recombinant protein. Other methods for confirming the expression in transformed cells may involve RT-PCR utilizing primers for a sequence mRNA or immunofluorescence techniques on transformed cells in culture.

Combination with Other Therapies

The biological electrode and methods of the invention may also be utilized in combination with other cardiac therapies when appropriate. In certain embodiments, drugs used to treat certain types of conduction defects can be administered in combination with implanting recombinant connexin cells into the damaged myocardium (e.g., prior to, during and/or after implantation). Cardiac drugs that are suitable for use in combination therapy with connexin or other gap junction enhancement methods include, but are not limited to, growth factors, polynucleotides encoding growth factors, angiogenic agents, calcium channel blockers, antihypertensive agents, antimitotic agents, inotropic agents, antiatherogenic agents, anti-coagulants, beta-blockers, anti-arrhythmic agents, antiinflammatory agents, vasodilators, thrombolytic agents, cardiac glycosides, antibiotics, antiviral agents, antifungal agents, agents that inhibit protozoans, antiarrhythmic agents (used for treatment of ventricular tachycardia), nitrates, angiotensin converting enzyme (ACE) inhibitors; brain natriuretic peptide (BNP); antineoplastic agents, steroids, and the like.

### Energy Harvesting Devices

An implantable device of the invention may include an energy harvesting device, e.g., a kinetic microelectrode, or those disclosed in U.S. application Serial No. 11/469,018, entitled “SENSOR FOR EDEMA”, filed Aug. 31, 2006, and 11/466,974, entitled “INTEGRATED CARDIAC RHYTHM MANAGEMENT SYSTEM WITH HEART VALVE”, filed on Aug. 24, 2006, assigned to Cardiac Pacemakers, Inc., the disclosures of which are incorporated by reference herein.

### Example of Electrical Stimulation System Including Biological Electrode

A biological electrode including donor cells embedded in an electrically conductive polymeric matrix provides for a stimulation threshold that is substantially lower than a stimulation threshold associated with a metal electrode. The stimulation threshold is the minimum energy level required to evoke action potentials in cells using electrical stimulation, such as cardiac pacing and neurostimulation. The electrically conductive polymeric matrix is a “three-dimensional network” that has a thickness allowing for multiple layers of the donor cells to be embedded. The donor cells are of the type that form or are capable of forming gap junctions with cells, e.g., cells in a tissue, of a recipient mammal (such as cardiomyocytes or neurons) following the placement of the biological electrode in the recipient, e.g., on a tissue of the recipient. In response to an electrical stimulation pulse delivered to the biologic electrode, the embedded donor cells are depolarized. The gap junctions function as a biological bridge for transmitting the action potential evoked in the donor cells to the cells of the tissue to activate the tissue, thereby causing tissue responses such as myocardial contractions. The stimulation threshold is thus the minimum energy required to depolarize the donor cells embedded in the electrically conductive polymeric matrix.

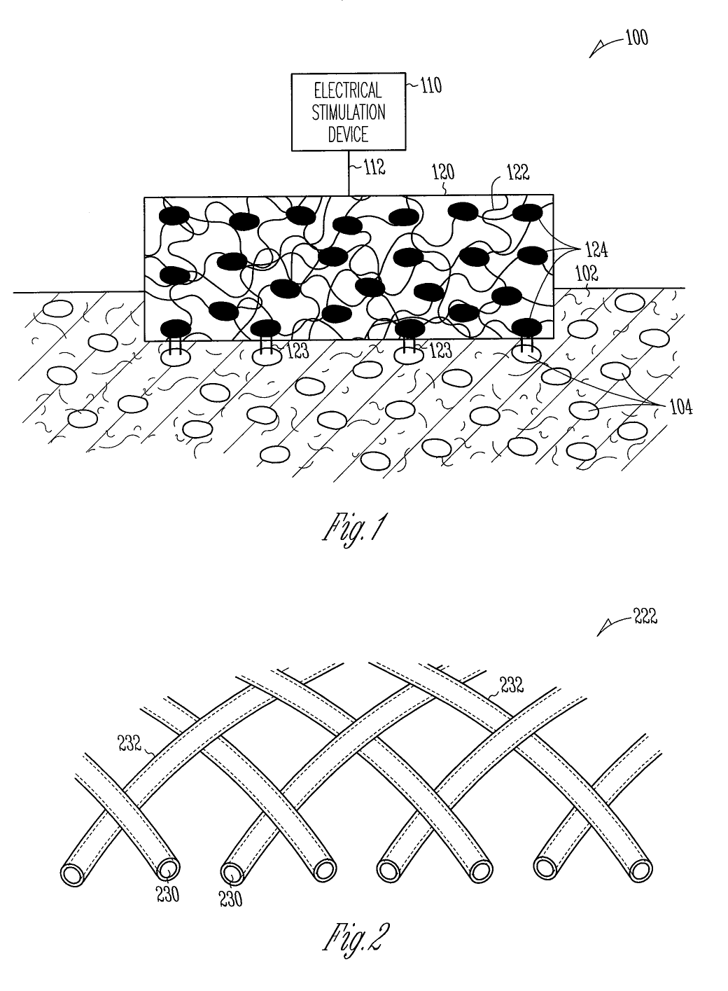

FIG. 1 is an illustration of an embodiment of an electrical stimulation system 100 and portions of an environment in which system 100 operates. System 100 includes an electrical stimulation device 110 connected to a biological electrode 120 via an electrical couple 112. In one embodiment, system 100 is an implantable system, with electrical stimulation device 110 being an implantable medical device. In one embodiment, electrical couple 112 includes an implantable lead.

Biological electrode 120 includes an electrically conductive polymeric matrix 122 in which donor cells 124 are embedded. Electrically conductive polymeric matrix 122 is biocompatible. To be chronically implantable, in one embodiment, electrically conductive polymeric matrix 122 is nonbioabsorbable, is nonbiodegradable, and is long-term failure/fatigue resistant. In one embodiment, biological electrode 120 has a thickness between 20 microns up to 10 to 900 mm. Biological electrode 120 is configured and tested for delivering electrical stimulation energy in the intended biological environment, in a way that prevents the conduction of action potentials from being disrupted by the body fluids after implantation.

In one embodiment, electrically conductive polymeric matrix 122 is a network of polymeric fibers that allows many contacts to be formed between the electrically conductive polymer and each of the donor cells. These contacts provide for a low threshold of energy required to electrically excite the donor cells by delivering electrical stimulation pulses to biological electrode 120.

Biological electrode 120 is configured to allow formation of gap junctions 123 between donor cells 124 and cells 104 of tissue, e.g., stimulated tissue, 102 after the biologic electrode is placed on the tissue. An interface between biological electrode 120 and tissue 102 is formed following the placement of biological electrode 120 on tissue 102 to allow transmission of the action potentials from donor cells 124 to cells 104. After biological electrode 120 is placed on tissue 102, the interface allowing transmission of the action potentials forms naturally, for instance within 12 hours, after which electrical stimulation device can deliver electrical stimulation pulses.

In various embodiments, tissue 102 represents any tissue that is excitable by the propagation of action potentials, such as tissue of the myocardium or nerves. Biological electrode 120 includes any biological electrode discussed in this document. Electrically conductive polymeric matrix 122 is formed by any suitable biocompatible polymer(s) discussed in this document. Donor cells 124 include any type(s) of donor cells discussed in this document.

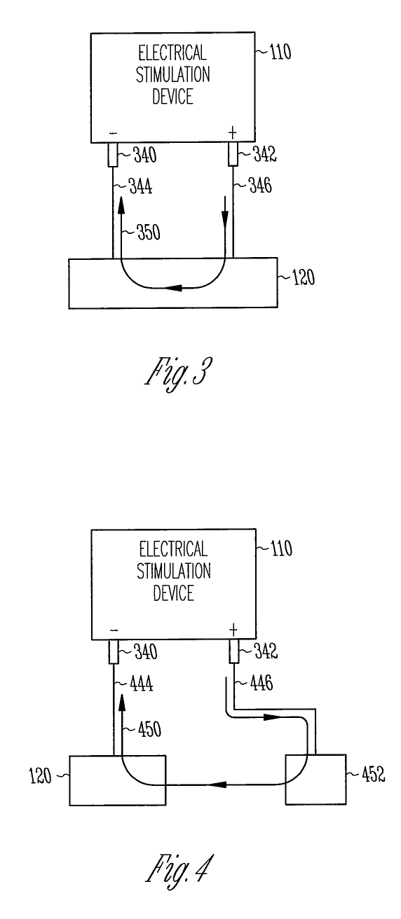

FIG. 2 is an illustration of an embodiment of an electrical conductive polymeric matrix 222. Electrical conductive polymeric matrix 222 represents a specific embodiment of electrical conductive polymeric matrix 122 and includes a first component 230 and a second component 232. First component 230 is a scaffold formed by a support network of polymeric fibers. Second component 232 is an electrically conducting polymeric layer formed on the polymeric fibers of first component 230. First component 230 provides the structural support for second component 232, which provides the electrical conductivity of electrically conductive polymeric matrix 222.

In one embodiment, electrical conductive polymeric matrix 222 is formed by co-polymerizing first component 230 and second component 232. In another embodiment, first component 230 is polymerized first to form the network of the polymeric fibers. Then, second component 232 is polymerized on the polymeric fibers of first component 230.

FIG. 3 is an illustration of an embodiment of electrical stimulation device 110 electrically coupled to biological electrode 120. Electrical stimulation device 110 includes at least one pair of output terminals 340 and 342 for delivering electrical stimulation pulses. Output terminal 340 is coupled to biological electrode 120 using an electrical conductor 344. Output terminal 342 is coupled to biological electrode 120 using an electrical conductor 346. When an electrical stimulation pulse is delivered from output terminals 340 and 342, electrical current flows through a path 350 as illustrated.

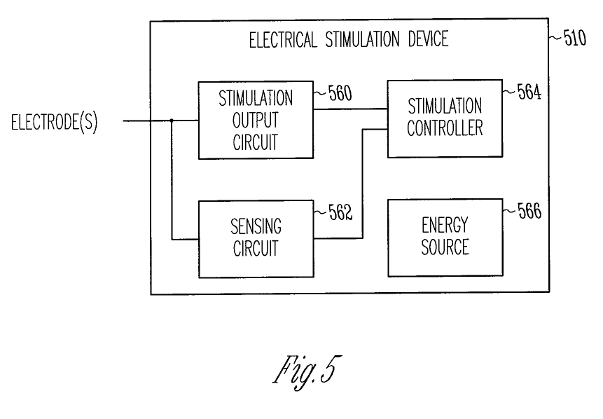

FIG. 4 is an illustration of another embodiment of electrical stimulation device 110 coupled to biological electrode 120 and another electrode 452. Output terminal 340 is coupled to biological electrode 120 using an electrical conductor 444. Output terminal 342 is coupled to electrode 452 using an electrical conductor 446. Examples of electrode 452 include another biological electrode and a metallic electrode such as a metal-plate electrode or a metal electrode formed on an implantable lead. In one embodiment, electrode 452 is formed by a portion of the housing of electrical stimulation device 110. When an electrical stimulation pulse is delivered from output terminals 340 and 342, electrical current flows through a path 450 as illustrated.

FIG. 5 is a block diagram illustrating an embodiment of portions of a circuit of an electrical stimulation device 510. Electrical stimulation device 510 represents a specific embodiment of electrical stimulation device 110 and includes a stimulation output circuit 560, a sensing circuit 562, a stimulation controller 564, and an energy source 566. Examples of electrical stimulation device 510 includes an implantable pacemaker, an implantable neurostimulator, any implantable device that evokes action potential in a body using electrical stimulation, and a portion thereof.

Stimulation output circuit 560 delivers electrical stimulation pulses such as cardiac pacing pulses or neurostimulation pulses to one or more electrodes, including biological electrode 120. Sensing circuit 562 senses physiological signals such as cardiac signals or neural signals using electrodes. Stimulation controller 564 controls the delivery of the electrical stimulation pulses. Energy source 566 provides the circuit of electrical stimulation device 510 with energy for its operation. In one embodiment, energy source 566 includes a battery. In another embodiment, energy source includes an energy harvesting device.

Biological electrode 120 may be used in any application where an electrical stimulation therapy is delivered to evoke action potentials in living tissue and a low stimulation threshold is desired. In various embodiments, a reduced stimulation threshold allows for miniaturization of an implantable device because of the reduced battery volume and/or increased longevity of the implantable device.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A biological electrode comprising a first biocompatible polymer, an electrically conducting biocompatible polymer and mammalian donor cells.

2. The biological electrode of claim 1 wherein the first biocompatible polymer is configured to provide structural support for the biological electrode.

3. The biological electrode of claim 2 wherein the electrically conducting biocompatible polymer is formed on the first biocompatible polymer.

4. The biological electrode of claim 3 comprising an electrically conductive polymeric matrix formed by the first biocompatible polymer and the electrically conducting biocompatible polymer, and wherein the donor cells are embedded in the electrically conductive polymeric matrix.

5. The biological electrode of claim 1 wherein the donor cells are capable of depolarization in response to a current.

6. The biological electrode of claim 1 wherein the donor cells are capable of differentiating into cells that depolarize in response to a current.

7. The biological electrode of claim 1 wherein the donor cells are stem cells.

8. The biological electrode of claim 1 wherein the donor cells are mesenchymal stem cells.

9. The biological electrode of claim 1 wherein the electrically conducting biocompatible polymer is nonbiodegradable.

10. The biological electrode of claim 1 wherein the donor cells are genetically altered.

11. The biological electrode of claim 1 wherein the donor cells are capable of forming gap junctions.

12. An electrical stimulation system for delivering electrical stimulation into target tissue including target cells, the system comprising:
a biological electrode including an electrically conductive polymeric matrix and mammalian donor cells embedded in the electrically conductive polymeric matrix; and
an electrical stimulation device electrically coupled to the biological electrode, the electrical stimulation device including a stimulation output circuit adapted to deliver electrical stimulation pulses capable of depolarizing the donor cells.

13. The system of claim 12 wherein the electrically conductive polymeric matrix comprises a network of fibers of a first biocompatible polymer configured to provide structural support for the biological electrode and an electrically conducting biocompatible polymer formed on the fibers of the first biocompatible polymer.

14. The system of claim 13 wherein the biological electrode is configured to allow action potentials to transmit from the donor cells to the target cells through gap junctions formed following placement of the biological electrode on the target tissue.

15. The system of claim 14 wherein the electrical stimulation device comprises an implantable cardiac pacemaker.

16. The system of claim 14 wherein the electrical stimulation device comprises an implantable neurostimulator.

17. The system of claim 14 wherein the electrical stimulation device comprising an energy source including an energy harvesting device.

18. A method to treat cardiac dysfunction, comprising introducing the biological electrode of claim 1 to a mammal.

19. The method of claim 18 wherein the mammal is a human.

20. The method of claim 18 wherein the donor cells are autologous.

21. The method of claim 18 wherein the donor cells are exogeneic or allogeneic.

22. The method of claim 18 wherein the biological electrode is connected to one or more leads.

23. The method of claim 18 wherein the dysfunction is sinus node dysfunction.

24. The method of claim 18 wherein the biological electrode comprises an electrically conductive polymeric matrix formed by the first biocompatible polymer and the electrically conducting biocompatible polymer, and the mammalian donor cells are embedded in the electrically conductive polymeric matrix.

25. The method claim 24 wherein introducing the biological electrode comprises introducing an electrical stimulation system into the mammal for delivering electrical stimulation into target tissue including target cells in the mammal, the electrical stimulation system including the biological electrode and an electrical stimulation device electrically coupled to the biological electrode, the electrical stimulation device including a stimulation output circuit adapted to deliver electrical stimulation pulses capable of depolarizing the donor cells.
